(19) United States
(12) Patent Application Publication
(10) Pub. No.: US 2006/0252714 A1
(43) Pub. Date: Nov. 9, 2006

(54) Pharmaceutical compositions and methods for treating multidrug resistant cancer

(76) Inventors: Stuart A. Berger, Toronto (CA); Yicheng Zhang, Toronto (CA)

Correspondence Address: BERESKIN AND PARR, 40 KING STREET WEST, BOX 401, TORONTO, ON, M5H 3Y2, CA

(21) Appl. No.: 10/542,602

(22) PCT Filed: Jan. 19, 2004
(86) PCT No.: PCT/CA04/00033

§ 371 Date: May 24, 2006

Related U.S. Application Data
(60) Provisional application No. 60/460,397, filed on Apr. 7, 2003.

(30) Foreign Application Priority Data
Jan. 17, 2003 (CA) 2416799

(51) Int. Cl. A61K 31/704; A61K 31/192

(52) U.S. Cl. 514/34; 514/569; 514/570

(57) ABSTRACT

The invention relates to a pharmaceutical composition comprising i) ketotifen or an analog thereof and ii) a chemotherapeutic drug subject to multi-drug resistance by P-gp, such as doxorubicin or an analog thereof. The pharmaceutical composition is useful for treating cancer. The pharmaceutical composition is also useful for i) preventing or treating multi-drug resistance in a subject or ii) preventing a chemotherapeutic drug subject to multi-drug resistance by P-gp induced cardiac tissue damage in a subject, such as doxorubicin or an analog thereof. The invention relates to a pharmaceutical composition comprising i) ketotifen or an analog thereof and ii) a chemotherapeutic drug subject to multi-drug resistance by P-gp, such as doxorubicin or an analog thereof. The pharmaceutical composition is useful for treating cancer. The pharmaceutical composition is also useful for i) preventing or treating multiamrg resistance in a subject or ii) preventing a chemotherapeutic drug subject to multi-drug resistance by P-gp induced cardiac tissue damage in a subject, such as doxorubicin or an analog thereof.

### FIELD OF THE INVENTION

The invention relates to pharmaceutical compositions and methods for chemotherapy. The invention reverses multidrug resistance to chemotheraputic agents and prevents cardiac damage caused by chemotheraputic agents.

### BACKGROUND OF THE INVENTION

Intrinsic or acquired resistance to chemotherapeutic agents is a major contributing factor to failure in cancer treatment. Clinical drug resistance often presents as a multi-drug resistance (MDR) phenotype, characterized as de novo resistance to a variety of structurally diverse cytotoxic drugs or as developed cross-resistance to chemotherapeutic agents that have never been used in previous chemotherapy [17]. Although the cellular basis underlying drug resistance is not fully understood, several factors have been identified that contribute to its development. These include drug efflux mechanisms, increased drug inactivation (e.g. glutathione-S-transferase and resistance to alkylating agents), drug target mutation (topoisomerase mutation), altered DNA repair and resistance to apoptosis (p53 mutation, bcl-2 overexpression etc.) [1]. Clinical drug resistance may be caused by any one or a combination of these mechanisms. Increased transmembrane efflux of xenobiotics is one of the best characterized mechanisms of MDR and is known to be mediated through over-expression of adenosine triphosphate (ATP)-binding cassette (ABC) transporter superfamily members such as P-glycoprotein (P-gp/MDR1), multidrug resistance associated protein (MRP1), or breast cancer resistance protein (BCRP) [5, 14, 19, 20]. P-gp, the most extensively studied of these transporters, is encoded by the mdr1 gene and found to be overexpressed in many tumor cells, including a variety of leukemias and solid tumors [30]. P-gp over-expression provides protection against a number of chemotheraeutic agents including anthracyclines, vinca alkaloids, antharacenes, camptothecin derivatives, epipodophyllotoxins, and tubulin polymerizing drugs [31].Transfection of the mdr1 gene to drug-sensitive cell lines can transfer the MDR phenotype [28]. In about 30-40% of primary and more than 50% of metastatic breast cancer patient samples, P-gp was overexpressed [16, 27]. Increased expression of P-gp correlates with adverse prognosis and is associated with poor chemotherapy response and overall survival [27].

The prognostic importance of P-gp overexpression shows that the ability to prevent or reverse multi-drug resistance would be clinically valuable. This has led to the identification of a wide variety of compounds that are capable of reversing MDR through the inhibition of P-gp. Preclinical in vitro and in vivo studies in mice using MDR reversing agents such as verapamil, quinidine and cyclosporine A have demonstrated enhanced anti-MDR tumor activity [9]. To date, clinical trials have been conducted to evaluate the efficacy of MDR reversing agents with mixed results. In some cases, serum levels of reversing agents needed to block P-gp could not be achieved. In other cases, P-gp could be blocked but the levels of chemotherapeutic drugs had to be reduced in order to prevent excessive toxicity. However, some small scale studies in P-gp positive AML and VAD-refractory multiple myeloma showed that incorporation of verapamil or cyclosporin in chemotherapy significantly improved overall survival [6, 18, 26]. New, more potent P-gp inhibitors such as PSC388, GF120918, dexverapamil and XR9576 are also currently being evaluated in clinical trials and to date, preliminary results indicate that at minimum, it is possible to obtain serum levels of reversing agents sufficient to block P-gp [22, 24]. However, there remains a need for compositions that more effectively prevent multi-drug resistance. As mdrl/P-gp is also expressed in certain normal tissues, blockade of P-gp in vivo by reversal drugs inevitably changes drug distribution and metabolism, thus altering the pharmacokinetics of chemotherapeutic agents. As a result, increased accumulation of the drugs in plasma or tissue can cause increased toxicity (Table 1).

TABLE I
Cardiotoxocity of chemotherapy

| Drug | Toxic dose range^{a} | Toxicity |
|---|---|---|
| Amsacrine | Conventional dose | Ventricular arrhythmia |
| Bisulfan | Oral daily dose | Endocardial fibrosis |
| Cisplatin | Conventional dose | Acute myocardial Ischemia |
| Cyclo- | >100-120 mg/kg | Congestive heart failure, |
| phosphamide | over 2 d | hemorrhagic myocarditis/ |
| | | pericarditis/necrosis |
| Daunorubicin | >550 mg/m² | Same toxocity as doxorubicin |
| | (total dose) | |
| Doxorubicin | >550 mg/m² | Congestive heart failure |
| | (total dose) | (cumulative toxic effect). |
| | | arrhythmias |
| | <550 mg/m² | Cardiac toxicity in the |
| | (total dose) | presense of additional |
| | | risk factors |
| Fluorouracil | Conventional dose | Angina/myocardial infarction |
| Interferons | Conventional dose | Exacerbation of underlying |
| | | cardiac disease |
| Interleukin-2 | Conventional dose | Acute myocardial injury, |
| | | ventricular arrhythmia, |
| | | hypotension |
| Mitomycin | Conventional dose | Myocardial damage similar to |
| | | radiation-induced injury |
| Mitoxantrone | >100-140 mg/m² | Congestive heart failure, |
| | (total dose) | decreased left-ventricular |
| | | equation fraction |
| Paclitaxel | Conventional dose | Bradycardia |
| Vinblastine | Conventional dose | Myocardial Infarction |
| Vincristine | Conventional dose | Myocardial Infarction |

^{a}Route of administration is IV unless otherwise indicated. Conventional dose is commonly accepted therapeutic range.
Adapted with permission from Grever M R, Grieshaber C K: Toxicology by organ system, in Holland J F et al {eds: Cancer Medicine, 4th ed, p. 897, Baltimore, Williams & Wilkins, 1997.

Doxorubicin, one of the most potent chemotherapeutic agents for treating hematological malignancies and solid tumors, has dose-limiting cardiotoxicity both in animal models and in cancer patients. In one study, coadministration of cyclosporin and doxorubicin resulted in 55% and 350% increase of area-under-the-curve (AUC) of doxorubicin and its metabolite doxorubicinol respectively (Bartlett, 1994). PSC388, when used in combination with doxorubicin, increased doxorubicin AUC by 10-fold [12]. Using a murine model, Sridhar showed that the combination of verapamil and doxorubicin increased peak doxorubicin concentration in heart tissue by about 40% compared to doxorubicin alone. This increased tissue doxorubicin level led to severe heart damage and significantly lower survival rate [25]. There is a need for compositions and methods for chemotherapy which do not cause heart damage. The need applies to the most common chemotherapy drugs, such as those shown in Table 2.

TABLE 2
Cytotoxic drugs which are transported by P-gp.

Anthracyclines
Doxorubicin
Daunorubicin
Idarubicin
Epirubicin
Epipodophyllotoxins
Etoposide
Teniposide
Taxanes
Paclitaxel
Docetaxel
Vinca Alkaloids
Vincristine
Vinblastine
Vinorelbine
Others
Mitoxantrone
Dactinomycin
Amsacrine
Trimetrexate
Mitomycin
Mithramycin

### SUMMARY OF THE INVENTION

The invention relates to a pharmaceutical composition comprising i) ketotifen or an analog thereof and ii) a chemotherapeutic drug subject to multi-drug resistance by P-gp, preferably an anthracycline, more preferably doxorubicin or an analog thereof. The pharmaceutical composition of the present invention is useful for treating cancer. The pharmaceutical composition is also useful for i) circumventing or treating multi-drug resistance in an animal or ii) preventing a chemotherapeutic drug subject to multi-drug resistance by P-gp, preferably anthracycline, more preferably doxorubicin or an analog thereof induced cardiac tissue damage in an animal. The invention also includes kits containing these compositions and methods of use of these pharmaceutical compositions. In variations of the invention, mitoxantrone, VP-16 and vinblastine, or analogs thereof, are useful in the compositions and methods of the invention in place of doxorubicin. Other useful compounds which are subject to P-gp-mediated efflux (preferably those compounds in the same class that have similar activities as doxorubicin, mitoxantrone, VP-16 and vinblastine and which do not cause cause cardiotoxicity) are described below. Ketotifen and its analogs or compounds such as cetirizine and mizolastine are also used with chemotherapy drugs described in this application (or analogs thereof).

The invention relates to a method for treating cancer, comprising treating or incubating cancer cells with a composition that is a Ca²⁺-mobilizing agonist while concurrently blocking Ca²⁺ influx, whereby the cancer cells are sensitized to cell death induction. I(etotifen is a first generation antihistamine with store-operated Ca²⁺ channel antagonist properties [10]. As a calcium influx blocker, it was previously demonstrated that ketotifen could induce cell death in an activation-enhanced manner in leukemia cells [13], mast cells [23], and breast cancer cells [29]. In the course of evaluating the ability of ketotifen to induce cell death in breast cancer cells, it was observed that ketotifen could sensitize multi-drug resistant human breast cancer cells to doxorubicin. The invention shows that ketotifen can reverse multi-drug resistance through inhibition of P-gp. More importantly, it shows that ketotifen also reduces cardiotoxicity caused by high dose doxorubicin ini vivo thus uniquely identifying ketotifen as both a MR-reversing and cardioprotective agent. Ketotifen restores sensitivity of P-glycoprotein-overexpressing, multi-drug resistant, MCF-7/adr cells to doxorubicin, mitoxantrone, VP-16 and vinblastine. hi vivo, it was demonstrated that pretreatment of mice with ketotifen caused an increased accumulation of doxorubicin in cardiac tissue, consistent with a block in drug clearance. However, it was also observed that unlike verapamil, ketotifen pre-treatment did not enhance doxorubicin toxicity but in fact provided protection, both at the level of cardiac tissue damage and in survival. The invention provides the surprising invention that ketotifen reverses multi-drug resistance due to P-glycoprotein overexpression and provides cardioprotection to doxorubicin.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

Preferred embodiments of the invention will be described in relation to the drawings in which:

FIG. 1 Dose response curve of ketotifen and verapamil as MDR reversal compounds. MCF-7/adr cells were treated with different concentrations of reversal compounds with or without 2 μM doxorubicin for 24 hours. Cells were harvested, and plated for growth of breast cancer colonies in triplicates. The data are presented as perent of control clonogenicity in the absence of drug (p<0.0001). DXN: doxorubicin.

FIG. 2 Influence of 10 μM of ketotifen on the toxicities of four cytotoxic drugs. MCF-7/adr cells were treated with different concentrations of chemotherapeutic agents in the presence or absence of 10 μM ketotifen for 24 hours. Cells were harvested, washed and plated for growth of breast cancer colonies as described in the Materials and Methods. The data are presented as perent of control clonogenicity in the absence of drug (p<0.0001). Ke: ketotifen.

FIG. 3 Ketotifen fails to reverse MDR of MCF-7/mx and MCF-7/vp cell lines. MCF-7/mx and MCF-7/vp cell lines were treated and assayed as described in FIGS. 1 and 2. The toxicities of mitoxantrone and Vp-16 were evaluated respectively in MCF-7/mx and MCF-7/vp cells. Ke: ketotifen.

FIG. 4 Flow Cytometric analysis of intracellular doxorubicin retention. MCF-7/adr cells (5×10⁵/ml) were incubated with 2 μg/ml of doxorubicin at 37° C. for 2.5 hours in the presence of different concentrations of ketotifen or verapamil. Cells were washed and resuspended in ice-cold PBS. Doxorubicin relative fluorescence was measured by flow cytometry. A: 0 μM, b: 2 μM, c: 10 μM ketotifen or verapamil.

FIG. 5 Doxorubicin accumulation in heart tissue. Mice were treated with i.p. injection of reveral agents ketotifen or verapamil, followed 30 minutes later by 15 mg/kg doxorubicin or saline (as control). Three hours following injection of doxorubicin, three mice were sacrificed in each group and the hearts were excised, rinsed,minced and homogenated. The tissue doxorubicin was extractd with ice-cold acid ethanol solution (0.3N HCl in 50% ethanol). The doxorubicin in the supernatants was measured by fluorescence spectrometry. DXN: doxorubicin, VPL: verapamil, Ke: ketotifen. **: p<0.01.

FIG. 6 Histological evaluation of cardiotoxicity. Mice were injected with reversal compounds ketotifen (25 mg/kg) or verapamil (25 mg/kg) followed by doxorubicin (15 mg/kg). Three mice in each group were sacrificed 4 days post treatment. The hearts were removed and fixed in 10% formalin. At least 3 sections were made and stained with H.E. Slides were evaluated by light microscopy (original magnification=400×). In mice receiving doxorubicin alone, some capillary dilation, degeneration and vacuolization can be readily observed. Combination of verapamil with doxorubicin aggravates these manifestations of cardiotoxicities especially the cytoplasmic vacuolization. Ketotifen, on the other hand, alleviates these pathological changes induced by doxorubicin.

FIG. 7 Modulation of doxorubicin toxicity by verapamil or ketotifen. Mice were injected with reversal compounds ketotifen (25 mg/kg) or verapamil (25 mg/kg) followed by doxorubicin (15 mg/kg). Mice were observed for survival for 30 days following treatment. ***: p<0.001

FIG. 8 Ketotifen extends survival in mulit-resistant P388/adr murine leukemia cells. Mice were injected with 5×10⁵ p388/adr cells and treated once per week with doxorubicin (4 mg/kg) preceeded by ketotifen (75 mg/kg 30 minutes prior). Both drugs were given intraperitoneal.

### DETAILED DESCRIPTION OF INVENTION

The inventors have shown that ketotifen can reverse multi-drug resistance through inhibition of P-gp. In addition, the inventors have shown that ketotifen reduces cardiotoxicity caused by high dose doxorubicin in vivo. Thus, the inventors have identified ketotifen as both a MDR-reversing and a cardioprotective agent. The invention relates to a pharmaceutical composition comprising i) ketotifen or an analog thereof and ii) a chemotherapeutic drug subject to multi-drug resistance by P-gp, preferably an anthracycline, more preferably doxorubicin or an analog thereof. The term subject to mutli-drug resistance means that the chemotherapeutic drug's efficacy is reduced, or may become reduced, in a subject, tissue or cell because of clinical drug resistance associated with multi-drug resistance. A person skilled in the art can assess whether a chemotherapeutic drug is subject to multi-drug resistance by P-gp. For example, the chemotherapeutic drugs to be tested can be incubated with cells, such as P388/adr murine leukemia cells and normal P388 cells as a control. If there is reduced killing of the P388/adr murine leukemia cells as compared to the controls (e.g. normal P388 cells or chemotherapeutic drugs known not to be subject to multi-drug resistance) then the chemotherapeutic agent is subject to multi-drug resistance. In order to determine whether the multi-drug resistance is by P-gp the cells can be stained with a P-gp antibody, such as MRK-16, and the expression of P-gp can be compared to controls. A person skilled in the art will appreciate that other cell lines could be used, such as comparing the toxicity of the chemotherapeutic agent and P-gp staining in normal MCR-7 cells versus MCF-7/adr cells.

The pharmaceutical composition of the present invention is useful for treating cancer. The pharmaceutical composition is also useful for i) circumventing or treating multi-drug resistance in an animal and ii) preventing a chemotherapeutic drug subject to multi-drug resistance by P-gp, preferably anthracycline, more preferably doxorubicin or an analog thereof induced cardiac tissue damage in an animal. The anthracycline protective effects and reversal of MDR were unknown prior to this invention.

The invention also includes a kit comprising the agents i) ketotifen or an analog thereof and ii) a chemotherapeutic drug subject to multi-drug resistance by P-gp, preferably anthracycline, more preferably doxorubicin or an analog thereof, and directions for administering i) and ii) to an animal, preferably for administering the agents to treat cancer, i) prevent or treating multi-drug resistance in an animal or ii) prevent a chemotherapeutic drug subject to multi-drug resistance by P-gp, preferably anthracycline, more preferably doxorubicin or an analog thereof induced cardiac tissue damage in an animal.

The invention also includes a method for treating cancer in an animal, comprising administering to the animal an effective amount of the pharmaceutical composition of the invention or the agents of the kit of the invention. The cancer can be a solid tumor or a hematological malignancy.

The invention also includes a method for i) circumventing or treating multi-drug resistance in an animal or ii) preventing a chemotherapeutic drug subject to multi-drug resistance by P-gp, preferably anthracycline, more preferably doxorubicin or an analog thereof induced cardiac tissue damage in an animal, comprising administering to the animal an effective amount of the pharmaceutical composition of the invention.

A preferred embodiment of the invention includes a method for treating cancer in an animal, including
administering to the animal an effective amount of ketotifen or an analog thereof, and
administering to the animal an effective amount of a chemotherapeutic drug subject to multi-drug resistance by P-gp, preferably anthracycline, more preferably doxorubicin or an analog thereof.

Preferably, the ketotifen is administered prior to the doxorubicin, more preferably at least 30 minutes prior to the doxorubicin. In the methods, the ketotifen or analog thereof and doxorubicin or analog thereof are preferably administered orally, intravenously, intraperitoneally, subcutaneously or rectally or by a combination of more than one of the foregoing.

The invention also includes the use of the pharmaceutical compounds and compositions of the invention as a pharmaceutical substance, preferably for treatment of multi-drug resistance.

In addition, the invention includes the use of the pharmaceutical compounds and compositions of the invention for preparation of a medicament, preferably for the treatment of multi-drug resistance.

Cancer

As mentioned above, one embodiment of the invention is a pharmaceutical composition for use in treating cancer. The invention also contemplates methods for treating cancer by administering compounds of the invention (for example, ketotifen and doxorubicin) to an animal. P-gp is found in a variety of leukemias and solid tumors [30]. The term cancer includes any cancer including, without limitation, ovarian cancer, pancreatic cancer, head and neck cancer, squamous cell carcinoma, gastrointestinal cancer, breast cancer (such as carcinoma, ductal, lobular, and nipple), prostate cancer, non small cell lung cancer, Non-Hodgkin's lymphoma, multiple myeloma, leukemia (such as acute lymphocytic leukemia, chronic lymphocytic leukemia, acute myelogenous leukemia, and chronic myelogenous leukemia), brain cancer, neuroblastoma, and sarcomas. [32-36] In a preferred, example the cancer cell overexpresses P-glycoprotein.

Ketotifen

Ketotifen has the chemical name 4-(1-Methyl-4-piperidylidene)-4H-benzo[4,5]cyclohepta[1,2-b] thiophen-10(9H)-one hydrogen fumarate and the molecular formula C₂₃H₂₃NO₅S (Chemical Abstracts Registry Number for Ketotifen is 34580-13-7). It is described, for example, in U.S. Pat. Nos. 3,682,930, 3,749,786, and 5,399,360, and German Patent 2, 111,071. The pharmacology, toxicology, metabolism and the clinical experience with ketotifen has been summarized by Sorlcin et al (Focus on Ketotifen. Ed. E. M. Sorkin. In Drugs, September 1990, vol. 40, no. 3, pp. 412-448). Examples of ketotifen analogs are found in U.S. Pat. Nos. 3,682,930 and 3,749,786. The preferred ketotifen analogs are those which are suitable for use in mammals, such as humans. Methods which may be employed in screening and identifying useful ketotifen analogs and derivatives are described, for example, in U.S. Pat. No. 3,749,786.

Doxorubicin

Doxorubicin has the chemical name 8S,10S)-10-[(3-amino-2,3,6-trideoxy-a-L-lyxo-hexopyranosyl)oxy]-8-glycolyl-7,8,9,10-tetrahydro-6,8,11-trihydroxy-1-methoxy-5,12-naphthacenedione hydrochloride. The molecular formula of the drug is C₂₇H₂₉NO₁₉.HCl and its analogs are also known in the art. Analogs include mitoxantrone, daunorubicin and N-acetyl daunorubicin. Other doxorubicin analogs are described in U.S. Pat. Nos. 4,672,057, 4,345,068, 4,314,054, 4,229,355, 4,216,157, 4,199,571, 4,138,480.

Other compounds are described below. Preferred compounds and analogs have at least 25%, 50%, more preferably at least 75% of the activity of doxorubicin and ketotifen for reversing MDR without cardiotoxicity. Activity may be measured by a MDR assay or cardioprotection study as described in this application.

“Preventing” or “Reversing” drug resistance means inhibiting P-gp to circumvent, reduce or avoid MDR. It does not necessarily mean modifying the cancer cells so that they no longer have the MDR phenotype of overexpressed P-gp.

Pharmaceutical Compositions

The above described substances may be formulated into pharmaceutical compositions for administration to animals in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Compositions for chemotherapy are described in Cancer Chemotherapy Handbook by David S. Fischer, et al. (5^{th} Ed., Mosby-Year Book, Inc.

Publication date, May, 1997); Lippincott's Cancer Chemotherapy Handbook by Delia C. Baquiran, Jean Gallagher (Lippincott Williams and Wilkins Paperback); Physician's Cancer Chemotherapy Drug Manual, 2002 and CD-ROM by Edward Chu, Vincent T. Devita (2002, Jones & Bartlett Pub)

Administration of a therapeutically active amount of pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of the substance to elicit a desired response in the individual. Dosage regimes may be adjusted to provide the optimum therapeutic response. For example, several divided doses may be administered daily or the dose may be proportionally reduced as indicated by the exigencies of the therapeutic situation.

An active substance may be administered in a convenient manner such as by injection (subcutaneous, intravenous, etc.), oral administration, rectal administration, inhalation, or transdermal application. Depending on the route of administration, the active substance may be coated in a material to protect the compound from the action of enzymes, acids and other natural conditions which may inactivate the compound.

The compositions described herein can be prepared by per se known methods for the preparation of pharmaceutically acceptable compositions which can be administered toanimals, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985) or Handbook of Pharmaceutical Additives (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids.

The above disclosure generally describes the present invention. A more complete understanding can be obtained by reference to the following specific examples. These examples are described solely for the purpose of illustration and are not intended to limit the scope of the invention. Changes in form and substitution of equivalents are contemplated as circumstances might suggest or render expedient. Although specific terms have been employed herein, such terms are intended in a descriptive sense and not for purposes of limitation.

### EXAMPLE 1

### Ketotifen Specifically Reverses MDR Mediated by P-pg Transporter

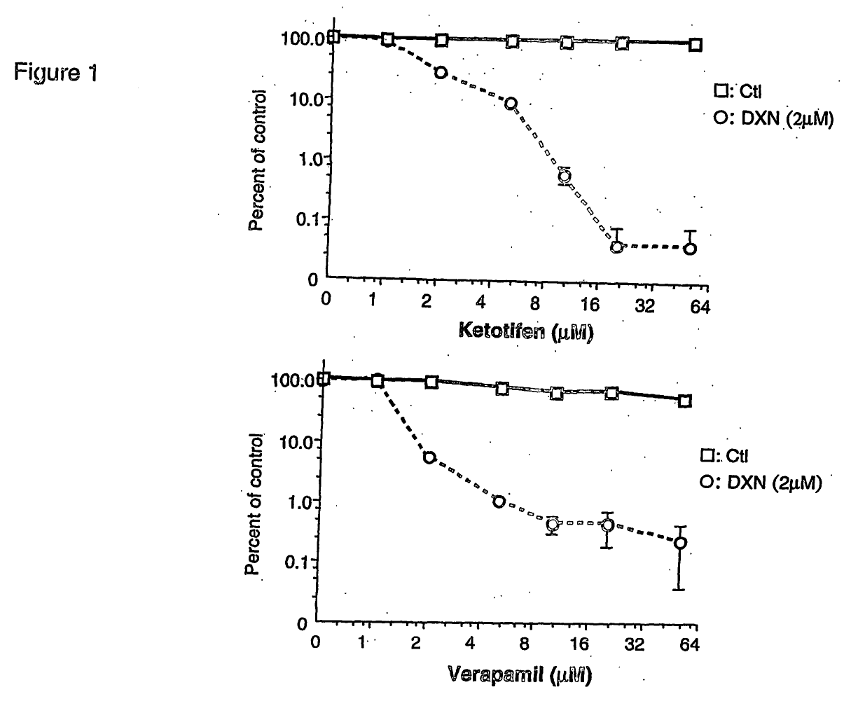

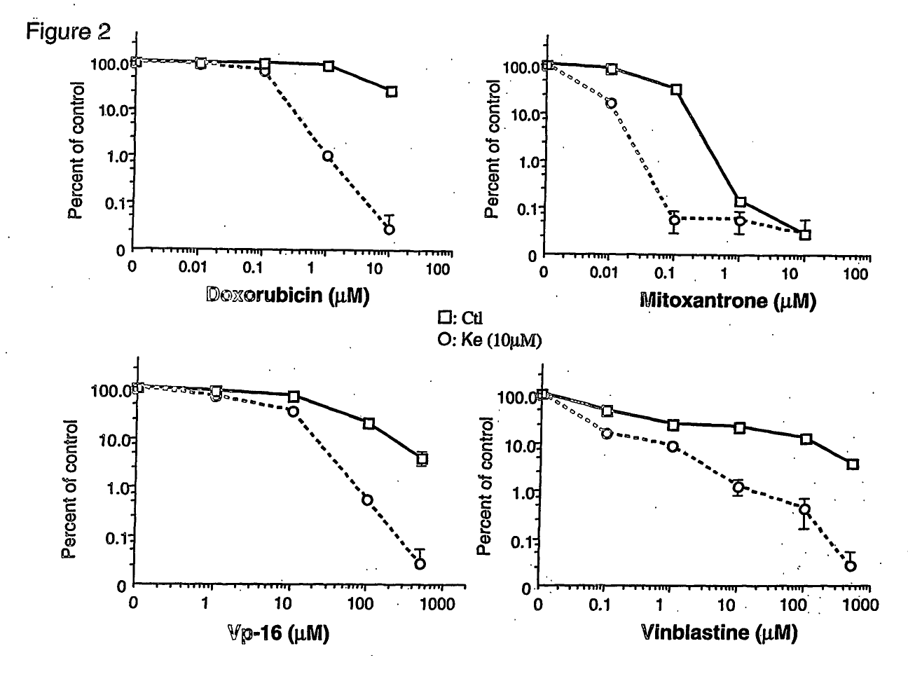

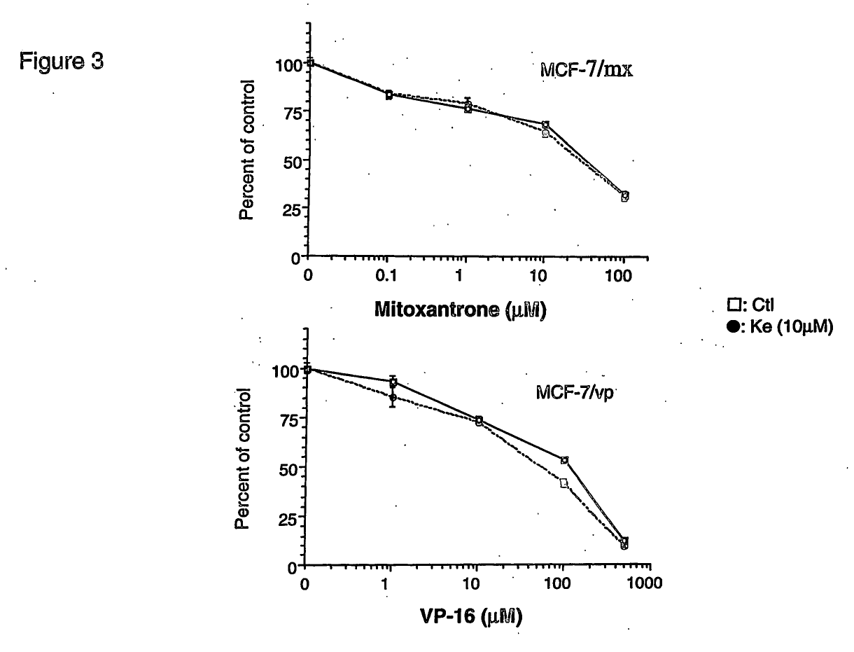

The toxicity of the cytotoxic drugs was measured by clonogenicity assay. As shown in FIG. 1A, significant dose-dependent reversal of doxorubicin resistance was observed with ketotifen. Beginning at 1 μM, ketotifen restored doxorubicin toxicity while at 10 μM, the MDR phenotype of MCF-7/adr cells was completely reversed. Over this concentration range, ketotifen itself is non-toxic to MCP-7/adr cells. The ability of ketotifen to restore sensitivity of MCF-7/adr cells to doxorubicin was compared with verapamil. As shown in FIGS. 1A and 1B, both ketotifen and verapamil reverse resistance at similar concentrations. MCF-7/adr cells are also relatively resistant to mitoxantrone, VP-16 and vinblastine. As shown in FIG. 2, the sensitivity to these drugs was also restored by 10 μM ketotifen. The IC₉₀s of different cytotoxic drugs were calculated from dose-response curves for MCF-7/adr or MCF-7/wt cells in the presence or absence of 10 mM of ketotifen. As summarized in Table 3, IC₉₀ levels on MCF-7/adr cells in the presence of ketotifen are almost identical to those for parental MCF-7 cells. In contrast to its reversing activity on MCF-7/adr cells, ketotifen influenced neither the toxicity of mitoxantrone on MCF-7/mx nor the toxicity of VP-16 on MCF-7/vp cells (FIG. 3). These two cell lines exhibit the MDR phenotype by overexpressing BCRP [19] and MRP transporters [21] respectively. Thus, ketotifen is a specific reversing agent for MDR associated with P-gp.

TABLE 3
IC₉₀ (μM) of cytotoxic drugs on MCF-7/wt and MCF-7/adr

MCF-7/adr

| | MCF-7/wt | −ke | +ke |
|---|---|---|---|
| Doxorubicin | 0.12 | >10 | 0.18 |
| Mitoxantrone | 0.01 | 0.22 | 0.02 |
| VP-16 | 17 | 220 | 20 |
| Vinblastine | 0.8 | 410 | 1.2 |

### EXAMPLE 2

### Increased Intracellular Retention of Doxorubicin in Ketotifen Treated MCF-7/adr Cells

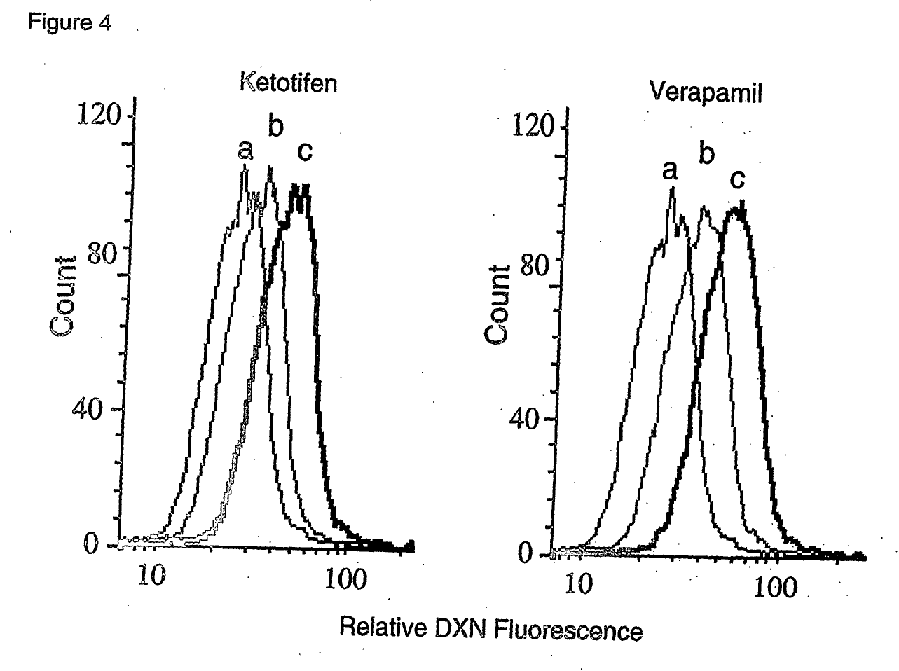

Most MDR reversing agents act by inhibiting the transporting activity of P-gp. In order to determine if ketotifen inhibits P-gp activity, the intrinsic fluorescence of doxorubicin was used as a marker and measured drug accumulation by flow cytometry. MCF-7/adr cells pretreated with ketotifen or verapamil were exposed to doxorubicin and fluorescence was measured. As shown in FIG. 4, in the presence of either verapamil or ketotifen, fluorescence from doxorubicin increased in the pre-treated cells. 2 μM of ketotifen increased relative fluorescence by 50%, while 10 mM of ketotifen nearly doubled the fluorescence intensity. This result shows that ketotifen causes an accumulation of doxorubicin in MCF-7/adr cells and that ketotifen mediates its reversal ability through the inhibition of drug efflux.

### EXAMPLE 3

### Tissue Doxorubicin Concentrations in the Heart

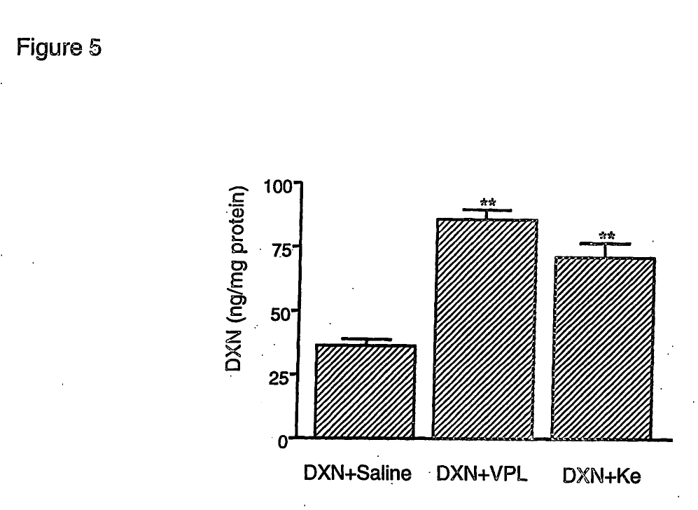

To determine the interactions of ketotifen with cytotoxic drugs in vivo, mice were given i.p. injections of reversal agent, followed by 15 mg/kg doxorubicin. Tissue concentrations of doxorubicin were determined by measuring doxorubicin fluorescence in heart tissue following different time periods after injection. The 3-hour time point values in different groups were compared as this point was the peak concentration was observed. As observed with verapamil, pre-treatment of mice with ketotifen significantly increased doxorubicin accumulation in the heart in comparison to control (72±5 vs 36±3 ng/mg protein, p<0.01, FIG. 5). This result shows that like verapamil, ketotifen causes a buildup of doxorubicin in tissue, likely due to inhibition of normal drug clearance mechanisms [25].

### EXAMPLE 4

### Ketotifen Prevents Cardiac Tissue Damage

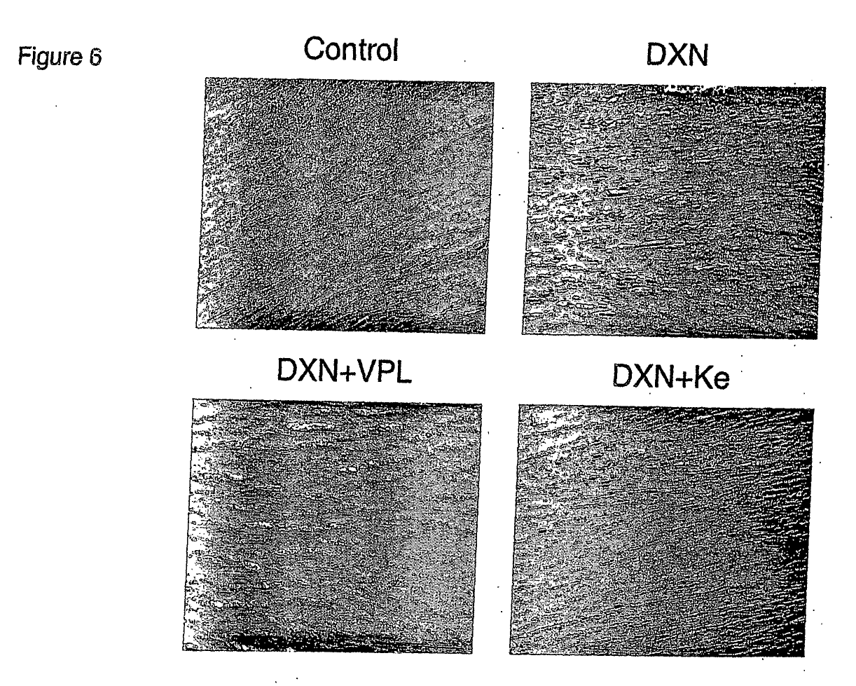

Cardiac tissue damage caused bv anthracyclines is well known and characterized by cardiac hypertrophy, vacuolization disruption of myofibrils and cell loss [3]. In order to characterize the effect of combined MDR reversing agent plus doxorubicin treatment on heart tissue, mice were treated with ketotifen or verapamil followed by doxorubicin. Four days later, heart tissue was fixed, sectioned and stained with hematoxylin and eosin. As shown in FIG. 6, mice treated with doxorubicin alone demonstrated well known pathological changes including dilation of capillaries, myocyte degeneration and vacuolization in left ventricular tissue (FIG. 6B). Addition of verapamil enhanced cardiac damage caused by doxorubicin (FIG. 6C). In contrast, heart tissue from mice pre-treated with ketotifen (25 mg/kg) 30 minutes before doxorubicin had observable decreases in the extent of cardiac damage (FIG. 6D) with less cell drop-out, maintenance of myofibril structure and less vacuolization.

### EXAMPLE 5

### Cardiotoxicity and Survival

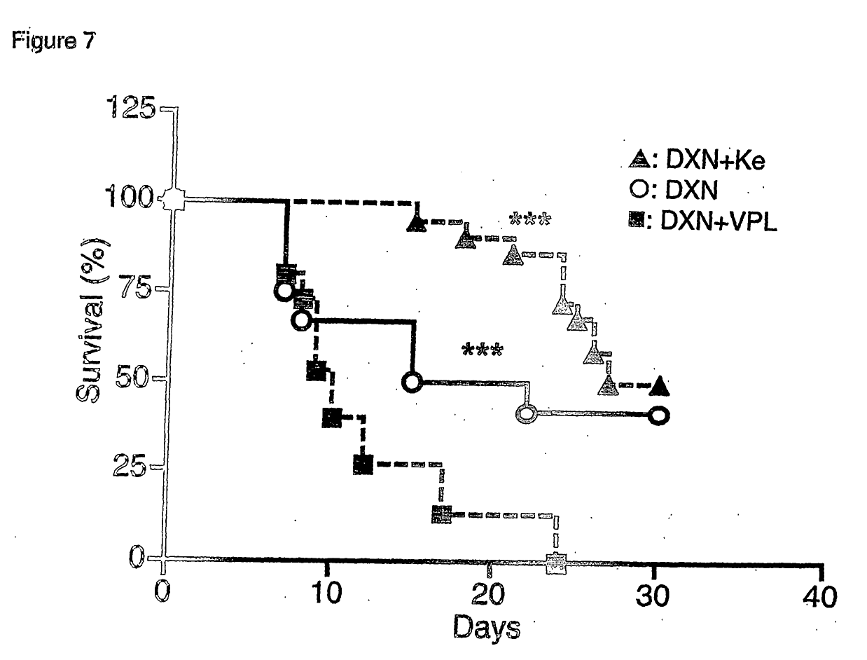

Since cardiac damage is reduced in mice receiving ketotifen plus doxorubicin, it was shown that the addition of ketotifen enhances mouse survival. Mice were pre-treated with ketotifen or verapamil, followed by a single treatment with doxorubicin and followed the animals over 5 weeks. Animals were sacrificed when they showed signs of lethargy or distress. As shown in FIG. 7, the survival rate of mice receiving doxorubicin plus verapamil was significantly lowered comparing to those mice treated with doxorubicin alone. For the doxorubicin plus verapamil group, survival rate at day 30 was 0% with median survival time of 12.3 days while 42% of the doxorubicin alone group survived 30 days post treatment with a median survival of 19.3 days (p<0.001). In contrast, pre-treatment of mice with ketotifen led to extended survival compared to doxorubicin alone with 57% survival rate at day 30 and a median survival time of 23.2 days (p<0.001 compared to Dox+Verapamil). Since prolongation of survival in mice treated with ketotifen plus doxorubicin correlated with the protection of ketotifen on the cardiotoxicity induced by doxorubicin, these results clearly show that ketotifen enhances survival due to cardioprotection.

In this study, it was shown that the antihistamine ketotifen can reverse multi-drug resistance in MCF-7/adr cells through inhibition of P-gp. This effect is specific in that cells overexpressing BCRP or MNP are not affected by ketotifen. At high concentrations, ketotifen also blocks store-operated Ca²⁺ influx and induces activation enhanced cell death [13, 29]. However, ketotifen's P-gp-inhibitory activity appears to be unrelated to its Ca²⁺ channel blocking activity since the concentrations required for P-gp inhibition are much lower. Furthermore, it was observed that Ca²⁺ ionophores have no effect on the ability of ketotifen to reverse MDR (Zhang and Berger; unpublished), providing additional evidence that ketotifen's MDR reversing activity is unrelated to its Ca²⁺ channel antagonism. It was further shown that pretreatment with ketotifen caused an increased accumulation of doxorubicin in mouse cardiac tissue, consistent with a block in drug clearance. However, unlike verapamil, ketotifen did not enhance doxorubicin toxicity but in fact provided protection, both at the level of cardiac tissue damage and in survival. These observations therefore show that ketotifen is unique in its ability to both reverse multi-drug resistance due to P-glycoprotein overexpression and provide cardioprotection to doxorubicin.

Although the mechanism of cardiotoxicity caused by anthracyclines is not fully understood, it is generally believed that highly active reactive oxygen species (ROS) triggered by anthracycine metabolites may play a central role in the initiation of a series reactions leading to myocyte damage [11, 15]. While antioxidants have shown some promise as cardioprotective agents in vitro and in animal models, clinical trials have not yet provided consistent benefit [4, 8]. Furthermore, the concern arises that the systemic application of antioxidants may also limit the anti-tumor efficacy of doxorubicin. Previous studies investigating the role of mast cell activation products in anthracycline-mediated cardiotoxicity had demonstrated that ketotifen could reduce doxorubicin cardiotoxicity and improve overall survival in a murine model [2]. In another study, doxorubicin was shown to induce mast cell degranulaton and histamine release, consistent with a role for mast cell activation in enhancing cardiac damage [7]. The invention shows the protective effect of ketotifen. Ketotifen's beneficial effect on survival can be partly attributed to cardiac protection based on the observed decrease in severity of cardiac damage in mice pre-treated with ketotifen.

The clinical use of anthracyclines is limited by its cardiotoxicity. Furthermore, schemes employing multi-drug reversing agents typically require reductions in chemotherapeutic dose due to inhibition of drug clearance mechanisms. The observations showing ketotifen as a multi-drug reversing agent with cardioprotective activity shows that this unique combination of properties is clinically useful in the control of multi-drug resistant tumors.

Materials and Methods

Human Breast Cancer Cell Lines and Culture Conditions

MCF-7 (MCF-7/wt) and its multidrug resistant variant MCF-7/adr cells were used. MCF-7/mx and MCF-7/vp cell lines were also used. Other cell lines could also be used. MCF-7/mx cell line was generated through selection in vitro with mitoxantrone and overexpresses Breast Cancer Resistance Protein (BCRP) [19]. The MCF-7/vp cell line was selected with etoposide and overexpresses Multidrug Resistance-associated Protein gene (MRP) [21]. All the cell lines were grown routinely as monolayer culture in Dulbecco's Minimal Essential Media (DME) supplemented with L-glutamine (2 mM), penicillin, streptomycin and 10% heat-inactivated fetal bovine serum SUBS, GIBCO) in an atmosphere of 5% CO₂ at 37° C. The cell lines were passaged weekly.

Chemicals.

Ketotifen, verapamil and all the chemotherapeutic agents (doxorubicin, etoposide VP-16, vinblastine and mitoxantrone) were purchased from Sigma Chemical Co. (St. Louis, Mo.). Ketotifen was freshly dissolved in DMSO before use, diluted with culture medium and added to the plate at the indicated concentrations. The final concentration of solvent DMSO was always less than 0.1%. All the other drugs were dissolved either in DMSO (VP-16 and vinblastine) or saline and stored at −20° C. as stock solutions.

Drug Treatments and Breast Cancer Clonogenic Assay.

Exponentially growing MCF-7/wt and its three mutants MCF-7/adr, MCF-7/mx and MCF-7/vp were trypsinized, washed with fresh medium and plated in 6-well plates at a density of 1×10⁵/ml. Cytotoxic drugs of different concentrations were applied to cells in the presence or absence of ketotifen or verapamil for 24 hours. Both adherent and non-adherent cells were collected and washed with fresh medium. Cell aliquots (5×10³) were plated in 1 ml of 0.3% agar over 1 ml of of 0.5% agar underlayer prepared in IMDM containing 10% horse serum (GIBCO). The upper layer consisted of 20% FBS, 10 μg/ml of bovine insulin, 2.5 mg/ml of hydrocortisone, 5×10⁻⁷M of 17-b-estradiol (Sigma) and 50 ng/ml of EGF (R & D Systems). Colonies larger than 50 μm in size were scored after 14 days of incubation at 37° C. in a humidified atmosphere of 5% CO₂ in air.

Flow Cytometry.

As doxorubicin itself is a fluorescent substance, the doxorubicin content in MCF-7/adr cells can be measured with flow cytometry. Briefly, cells (5×10⁵/ml) were incubated with 2 μg/ml of doxorubicin at 37° C. for 2.5 hours with or without reversal agents, washed and resuspended in ice-cold PBS. Doxorubicin fluorescence was measured by flow cytometry using a FACStar Plus flow cytometer (FL2, emission at >570 nm,Becton Dickinson). 10⁴ cells were analysed for each sample.

Animals and in vivo Treatment.

Female Balb/c mice (8-10 weeks of age, 20-22 g of body weight) were purchased from Jackson Laboratory (Maine, USA). Protocols were approved by the Animal Care Committee of the University Health Network. Animals were divided into 6 groups of 15 to 20 mice each and received drug treatments as follows: saline, ketotifen 25 mg/kg, verapamil 25 mg/kg, doxorubicin 15 mg/kg plus saline, doxorubicin 15 mg/kg plus ketotifen 25 mg/kg, doxorubicin 15 mg/kg plus verapamil 25 mg/kg. All the treatments were administrated via i.p. After treatment, mice were kept in sterile environment for six to eight weeks. Acute toxicities and survival was observed for different treatment groups. Mice were sacrificed when they displayed lethargic behaviour or any signs of distress. Three mice from each group were sacrificed on day 4-post treatment. Hearts were removed immediately and fixed in 10% neutral buffered formalin. Tissue sections were made from heart tissue and stained with hematoxylin and eosin or with 1% Toluidine blue to identify mast cells. All the slides were evaluated by light microscopy for cardiac damage, mast cell density and degranulation.

Doxorubicin Concentrations in Heart Tissues.

3 to 5 mice in each group were treated with the same drug combinations used for survival. Three hours following injection of doxorubicin, mice were sacrificed. Doxorubicin concentrations in heart tissues were determined by flurometric detection of doxorubicin using the method of Sridhar [25]. Briefly, hearts were excised immediately, rinsed with ice cold normal saline, minced with scissors, and homogenized in ice cold ethanol- acid solution (0.3 N HCl in 50% ethanol) using a Polytron homogenizer. The homogenates were centrifuged at 20,000 g for 20 minutes at 4° C. Fluorescence of the supernatants was measured using a Tecan Spectrafluor (excitation wavelenth of 468, emission wavelenth of 590, Hewlett Packard). The doxorubicin standard curve was made by mixing known amounts of doxorubicin with heart tissue and processed using an identical protocol. The fluorescence of supernatant from cardiac tissue without doxorubicin served as background. The concentration of doxorubicin was normalized to total protein content of the same tissue.

Statistical Analysis.

All the colony data were analyzed by two-way analysis of variance (ANOVA), with differences between individual means determined by Bonferroni's post-tests. Data were expressed as means ±SEM. The Kaplan-Meier estimate was used to determine differences in the survival periods for mice following different drug combination treatments.

### EXAMPLE 6

### Evaluation of the Efficacy of Ketotifen in Enhancing Chemotherapy of Multidrug Resistant Leukemia Cells

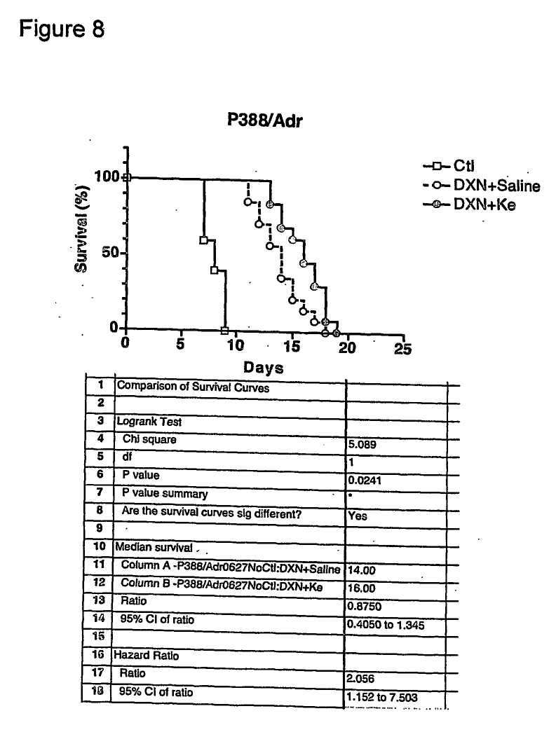

In vitro observations on the ability of ketotifen to reverse MDR, coupled with its ability to partially protect against doxorubicin cardiotoxicity, showed that the combination of ketotifen plus doxorubicin has superior anti-leukemic activity, particularly for multi-drug resistant disease. The combination of ketotifen plus doxorubicin was evaluated in the p388 multi-drug resistant leukemia model. This model has been used in a number of studies to assess efficacy of MDR reversing agents [43-47] and is a useful and rapid approach. Highly multi-drug resistant P388/adr murine leukemia cells were obtained NCI/Frederick Cancer DCT repository. Mice were inoculated with 5×10⁵ p388/adr cells and treated once per week with doxorubicin (4 mg/kg) preceded by ketotifen (75 mg/kg 30 minutes prior). Both drugs were given intraperitoneal. Ketotifen extended survival 2 days compared to doxorubicin alone (p=0.02). See FIG. 8. This is comparable to that observed with other drugs such as verapamil [47].

### EXAMPLE 7

### Evaluation of Tile Efficacy of Ketotifen to Reverse MDR and to Prevent Cardiac Damage in a Breast Cancer Model

Human multi-drug resistant breast cancer cells, such as MCR-7/adr cells, are implanted into immune defiant mice, such as female SCID/Rag2 m immunodeficient mice, to test the ini. vivo ability of ketotifen to reverse multi-drug resistance, coupled with its ability to protect against doxorubicin cardiotoxicity. The immunodeficiency of these mice prevents them from rejecting human tissue and these mice are therefore suitable hosts for xenographs.

MDR Reversal

On day 1, 1.5 mg Estrogen pellets are implanted at the base of the neck. On day 2, mice are inoculated with 10 million MCF-7/adr cells in a 0.1 ml volume subcutaneously in the flank. When the tumors are 5 mm in diameter, the mice (at least 4 per group) are treated with ketotifen (typically 75 mg/kg intraperitoneally) followed by doxorubicin (typically 4 mg/kg intiperitoneally) 30 minutes later. This treatment is repeated weekly and tumor size and body weight are examined over time.

Cardiac Function

The mice and their normal counterparts are challenged with a single i.p. injection of doxorubicin (15 mg/kg). One cohort of 10 mice from each group are evaluated for lethality, as defined by failure to thrive to the point where the animals have reduced mobility and are impaired for feeding. Reduced mobility is defined as decreased mobility such that nesting functions or other normal activity is decreased. Impaired feeding means that the mice are unable or uninterested in consuming solid food or cannot reach up to drink liquid from the suspended water bottle. A second cohort (5 mice per group) is evaluated for cardiac function. To be considered able to protect against cardiotoxicity the results must be statistically significant compared to the controls. Typically, hearts exposed to excessive levels of anthracyclines undergo cardiac hypertophy and cell loss [37,38]. This manifests itself in impaired heart function that can be evaluated by measuring cardiac function non-invasively and invasively as well as through pathological analysis of affected tissue. For non-invasive measurements, echocardiography is used. Echocardiography is a commonly used clinical tool in the management of patients undergoing anthracycline treatment [39,40]. At 4 days post-treatment, anesthetized mice are placed on a mouse pad equipped with a heater, continuous ECG output and rectal temperature monitoring. Transthoracic 2-dimensional, M-mode and Doppler echocardiographic examination is performed using an Acuson Sequoia C256 system. In addition, treated mice will undergo ex-vivo assessment of cardiac function. The hearts are isolated in a Langendorff preparation with an oversized intraventricular balloon at graduated incremental volumes to derive intrinsic pressure-volume (P-V) relationship, to determine load independent ventricular systolic function; and diastolic compliance P-V relationship to characterize diastolic relaxation [41]. Finally, heart tissue is analysed pathologically for apoptosis by TUNEL assay, fibrillar collagen content is determined using Picosirius Red staining in conjunction with light microscopy and videodensitometry, and collagen content are measured using hydroxyproline assay with Ehrlich's reagent and absorbance at 550 nm as previously described [42]. These measurements provide an accurate and quantitative picture of the extent of cardiomyopathy in these mice. The animals pretreated with ketotifen have significantly better heart function and less damage than those animals not pretreated with ketotifen.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended caims.

All publications, patents and patent applications are herein incorporated by reference in their entirety to the same extent as if each individual publication, patent or patent application was specifically and individually indicated to be incorporated by reference in its entirety.

### REFERENCES

1. Arceci R J (2000) Can multidrug resistance mechanisms be modified? Br J Haematol 110: 285
2. Bartoli Klugman F, Decorti G, Candussio L, Mallardi F, Grill V, Zweyer M, Baldini L (1988) Effect of ketotifen on adriamycin toxicity: role of histamine. Cancer Lett 39: 145
3. Birtle A J (2000) Anthracyclines and cardiotoxicity. Clin Oncol 12: 146
4. Blatt J (1997) ICRF-187 as a cardioprotectant in children treated with anthracyclines. Pediatr Hematol Oncol 14: iii
5. Cole S P, Deeley R G (1998) Multidrug resistance mediated by the ATP-binding cassette transporter protein MRP. Bioessays 20: 931
6. Dalton W S, Crowley J J, Salmon S S, Grogan T M, Laufman L R, Weiss G R, Bonnet J D (1995) A phase III randomized study of oral verapamil as a chemosensitizer to reverse drug resistance in patients with refractory myeloma. A Southwest Oncology Group study. Cancer 75: 815
7. Decorti G, Candussio L, Klugmann F B, Strohmayer A, Mucci M P, Mosco A, Baldini L (1997) Adriamycin-induced histamine release from heart tissue in vitro. Cancer Chemother Pharmacol 40: 363
8. Dorr R T (1996) Cytoprotective agents for anthracyclines. Semin Oncol 23: 23
9. Ford J M (1995) Modulators of multidrug resistance. Preclinical studies. Hematol Oncol Clin North Am 9: 337
10. Franzius D, Hoth M, Penner R (1994) Non-specific effects of calcium entry antagonists in mast cells. Pflugers Archiv European Journal of Physiology 428: 433
11. Gewirtz D A (1999) A critical evaluation of the mechanisms of action proposed for the antitumor effects of the anthracycline antibiotics adriamycin and daunorubicin. Biochem Pharmacol 57: 727
12. Giaccone G, Linn S C, Welink J, Catimel G, Stieltjes H, van der Vijgh W J, Eeltink C, Vermorken J B, Pinedo H M (1997) A dose-finding and pharmacokinetic study of reversal of multidrug resistance with SDZ PSC 833 in combination with doxorubicin in patients with solid tumors. Clin Cancer Res 3: 2005
13. Gommerman J L, Berger S A (1998) Protection from apoptosis by steel factor but not interleukin-3 is reversed through blockade of calcium influx. Blood 91: 1891
14. Gottesman M M, Pastan I (1993) Biochemistry of multidrug resistance mediated by the multidrug transporter. Annu Rev Biochem 62: 385
15. Horenstein M S, Vander Heide R S, L'Ecuyer T J (2000) Molecular basis of anthracycline-induced cardiotoxicity and its prevention. Mol Genet Metab 71: 436
16. Lehnert M (1998) Chemotherapy resistance in breast cancer. Anticancer Res 18: 2225
17. Ling V (1997) Multidrug resistance: molecular mechanisms and clinical relevance. Cancer Chemother Pharmacol 40: S3
18. Mross K, Bohn C, Edler L, Jonat W, Qucisser W, Heidemann E, Goebel M, Hossfeld D K (1993) Randomized phase II study of single-agent epirubicin ±verapamil in patients with advanced metastatic breast cancer. An AIO clinical trial. Arbeitsgemeinschaft Internistische Onkologie of the German Cancer Society. Ann Oncol 4: 45
19. Ross D D, Yang W, Abruzzo L V, Dalton W S, Schneider E, Lage H, Dietel M, Greenberger L, Cole S P, Doyle L A (1999) Atypical multidrug resistance: breast cancer resistance protein messenger RNA expression in mitoxantrone-selected cell lines. J Natl Cancer Inst 91: 429
20. Scheper R J, Broxterman H J, Scheffer G L, Kaaijk P, Dalton W S, van Heijningen T H, van Kalken C K, Slovak M L, de Vries E G, van der Valk P, et al. (1993) Overexpression of a M(r) 110,000 vesicular protein in non-P-glycoprotein-mediated multidrug resistance. Cancer Res 53: 1475
21. Schneider E, Horton J K, Yang C H, Nakagawa M, Cowan K H (1994) Multidrug resistance-associated protein gene overexpression and reduced drug sensitivity of topoisomerase II in a human breast carcinoma MCF7 cell line selected for etoposide resistance. Cancer Res 54: 152
22. Silcic B I, Fisher G A, Lum B L, Halsey J, Beketic-Oreskovic L, Chen G (1997) Modulation and prevention of multidrug resistance by inhibitors of P-glycoprotein. Cancer Chemother Pharmacol 40: S13
23. Soboloff J, Berger S A (2002) Sustained ER Ca2+ depletion suppresses protein synthesis and induces activation-enhanced cell death in mast cells. J Biol Chem 277: 13812
24. Spicer D, Arzoo K, Groshen S (2000) Valspodar +paclitaxel in advanced breast cancer: A California Cancer Consortium Phase II randomized trial. 19: 179a
25. Sridhar R, Dwivedi C, Anderson J, Baker P B, Sharma H M, Desai P, Engineer F N (1992) Effects of verapamil on the acute toxicity of doxorubicin in vivo. J Natl Cancer Inst 84: 1653
26. Tan B, Piwnica-Worms D, Ratner L (2000) Multidrug resistance transporters and modulation. Curr Opin Oncol 12: 450
27. Trock B J, Leonessa P, Clarke R (1997) Multidrug resistance in breast cancer: a meta-analysis of MDR1/gp170 expression and its possible functional significance. J Natl Cancer Inst 89: 917
28. Ueda K, Cardarelli C, Gottesman M M, Pastan I (1987) Expression of a full-length cDNA for the human “MDR1” gene confers resistance to colchicine, doxorubicin, and vinblastine. Proc Natl Acad Sci U S A 84: 3004
29. Zhang Y, Crump M, Berger S A (2002) Purging of contaminating breast cancer cells from hematopoiteic progenitor cell preparations using Activation Enhanced Cell Death. Breast Cancer Research and Treatment 72: 265
30. Juliano R L, Ling V (1976) A surface glycoprotein modulating drug permeabiligy in Chines hamster ovary cell mutants. Biochim Biophys Acta 455: 152-62.
31. Litman T, Druley T E, Stein W D, Bates S E (2001) From BDR to MXR: new understanding of multidrug resistance systems, their properties and clinical significance. Cell Mol Life Sci 58: 931-59.
32. Leith C. (1998) Multidrug resistance in leukemia. Curr Opin Hematol 5: 287-91.
33. Norgaard J M, Hokland P (200) Biology of multiple drug resistance in acute leukemia. Int. j. Hematol 72: 290-7.
34. Marie J P (2001) Drug resistance in hematologic malignancies. Curr Opin Oncol 13: 462-9.
35. Motoji T, Motomuara S, Wang Y H (2000) Multidrug resistance oa cute leukemia and a strategy to overcome it. Int J Hematol 72: 418-24.
36. Hegewisch-Becker S, Hossfeld D K (1996) The MDR phenotype in hematologic malignancies: prognostic relevance and future perspectives. Ann Hematol 72: 105-17.
37. Ferrans V J (1978) Overview of cardiac pathology in relation to anthracycline cardiotoxicity. Cancer Treat Rep 62:955-61.
38. Combs A B, Acosta D (1990) Toxic mechanisms of the heart: a review. Toxicol Pathol 18:583-96.
39. Birtle A J (2000) Anthracyclines and cardiotoxicity. Clin Oncol 12:146-52.
40. Singal P K, Iliskovic N (1998) Doxorubicin-induced cardiomyopathy. N Engl J Med 339:900-5.
41. Orenstein T L, Parker T G, Butany J W, Goodman J M, Dawood F, Wen W H, Wee L, Martino T, McLaughlin PR, Liu P P (1995) Favorable left ventricular remodeling following large myocardial infarction by exercise training. Effect on ventricular morphology and gene expression. J Clin Invest 96:858-66.
42. Lee J K, Zaidi S H, Liu P, Dawood F, Cheah A Y, Wen W H, Saild Y, Rabinovitch M (1998) A serine elastase inhibitor reduces inflammation and fibrosis and preserves cardiac function after experimentally-induced murine myocarditis. Nat Med 4:1383-91.
43. Ramu A, Spanier R, Rahamimoff H, Fuks Z (1984) Restoration of doxorubicin responsiveness in doxorubicin-resistant P388 murine leukaemia cells. Br J Cancer 50:501-7.
44. Boesch D, Gaveriaux C, Jachez B, Pourtier-Manzanedo A, Bollinger P, Loor F (1991) In vivo circumvention of P-glycoprotein-mediated multidrug resistance of tumor cells with SDZ PSC 833. Cancer Res 51:4226-33.
45. Watanabe T, Tsuge H, Oh-Hara T, Naito M, Tsuruo T (1995) Comparative study on reversal efficacy of SDZ PSC 833, cyclosporin A and verapamil on multidrug resistance in vitro and in vivo. Acta Oncol 34:235-41.
46. Noviello E, Allievi E, Russo P. Parodi S (1997) Effects of Dex-Verapamil on Doxorubicin cytotoxicity in P388 murine leukemia cells. Anticancer Drug Des 12:261-76.
47. Tsuruo et al. (1981) Cancer Research 41: 1967.

1. A pharmaceutical composition comprising i) ketotifen or an analog thereof and ii) a chemotherapeutic drug subject to multi-drug resistance by P-gp.

2. (canceled)

3. (canceled)

4. (canceled)

5. (canceled)

6. The pharmaceutical composition according to claim 1, wherein the chemotherapeutic drug subject to multi-drug resistance by P-gp comprises an anthracycline.

7. The pharmaceutical composition according to claim 1, wherein the chemotherapeutic drug subject to multi-drug resistance by P-gp comprises doxorubicin or an analog thereof.

8. A kit comprising the agents i) ketotifen or an analog thereof and ii) a chemotherapeutic drug subject to multi-drug resistance by P-gp, and directions for administering i) and ii) to an animal.

9. (canceled)

10. (canceled)

11. The pharmaceutical composition of claim 1 in an oral, intravenous, intraperitoneal, subcutaneal or rectal dosage form or in combination of the foregoing dosage forms.

12. (canceled)

13. (canceled)

14. (canceled)

15. (canceled)

16. (canceled)

17. (canceled)

18. (canceled)

19. (canceled)

20. A method for i) preventing or treating multi-drug resistance in an animal or ii) preventing a chemotherapeutic drug subject to multi-drug resistance by P-gp induced cardiac tissue damage in an animal, comprising administering to the animal an effective amount of the pharmaceutical composition of claim 14.

21. The method according to claim 20, wherein the chemotherapeutic drug subject to multi-drug resistance by P-gp comprises an anthracycline.

22. The method according to claim 20, wherein the chemotherapeutic drug subject to multi-drug resistance by P-gp comprises doxorubicin or an analog thereof.

23. A method for preventing or treating multi-drug resistance in cancer cells, tumors or neoplasia, comprising administering to the animal an effective amount of the pharmaceutical composition of claim 1.

24. The method according to claim 23, wherein the chemotherapeutic drug subject to multi-drug resistance by P-gp comprises an anthracycline.

25. The method according to claim 23, wherein the chemotherapeutic drug subject to multi-drug resistance by P-gp comprises doxorubicin or an analog thereof.

26. A method for treating cancer in an animal, comprising
administering to the animal an effective amount of ketotifen or an analog thereof, and
administering to the animal an effective amount of a chemotherapeutic drug subject to multi-drug resistance by P-gp.

27. The method according to claim 26, wherein the chemotherapeutic drug subject to multi-drug resistance by P-gp comprises an anthracycline.

28. The method according to claim 26, wherein the chemotherapeutic drug subject to multi-drug resistance by P-gp comprises doxorubicin or an analog thereof.

29. The method of claim 28, wherein the ketotifen is administered prior to the doxorubicin.

30. The method of claim 29, wherein the ketotifen is administered at least 30 minutes prior to the doxorubicin.

31. The method of 26 wherein ketotifen or analog thereof and doxorubicin or analog thereof are administered orally, intravenously, intraperitoneally, subcutaneally or rectally or by a combination of more than one of the foregoing.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. A method of determining whether cancer should be treated with ketotifen or an analog thereof and a chemotherapeutic drug subject to multi-drug resistance by P-gp, comprising determining whether P-gp is overexpressed by the cell.

46. (canceled)

47. (canceled)

48. (canceled)

49. (canceled)

50. (canceled)

51. (canceled)

52. (canceled)

53. The method of claims 45, further comprising treating the cancer with ketotifen or an analog thereof and a chemotherapeutic drug subject to multi-drug resistance by P-gp.

54. (canceled)

55. (canceled)

56. A method for i) preventing or treating multi-drug resistance in an animal or ii) preventing a chemotherapeutic drug subject to multi-drug resistance by P-gp induced cardiac tissue damage in an animal, comprising administering to the animal an effective amount of a compound having MDR-reversing and cardiac-protective activity.

57. (canceled)

58. (canceled)
